(19) United States
(12) Patent Application Publication
(10) Pub. No.: US 2012/0325690 A1
(43) Pub. Date: Dec. 27, 2012

(54) FLOSS TOOTHPICK PACKAGE

(76) Inventors: Ronald Brodkin, Boca Raton, FL (US); J. Randall Timko, Deland, FL (US)

(21) Appl. No.: 13/531,548

(22) Filed: Jun. 24, 2012

Related U.S. Application Data
(60) Provisional application No. 61/501,160, filed on Jun. 24, 2011.

(51) Int. Cl. A61B 19/02; A61C 15/00; B65B 11/48

(52) U.S. Cl. 206/63.5; 53/460

(57) ABSTRACT

A personal hygiene containing apparatus includes a folded matchbook-like envelope structure and at least one plurality dental cleaning devices detachably secured to the envelope structure. The outside of the envelope includes advertising materials. The dental cleaning devices may be floss toothpicks having a reflective surface and may be individually sealed.

### FIELD

The present invention relates to a package for dispensing a plurality of interdental cleaning devices. Particularly, the present invention provides a disposable packaging for floss toothpicks.

### BACKGROUND

It has been well known in the past to provide some form of an implement to facilitate the removing of food particles from between a person's teeth. Such items have been frequently referred to as a toothpick and generally take the form of an elongated pointed tool which is adapted to be inserted between a person's teeth and moved in order to dislodge any food particles and plaque located between the teeth.

It has been further found to be desirable to not only employ the use of a pointed instrument, but also to employ the use of a strand of thread or filament which is commonly referred to as dental floss. A segment of dental floss is stretched taut and then inserted between a person's teeth and moved back and forth in order to effect removal of any lodged food particles and plaque.

Over time, various persons have discovered that it is practical and useful to apply a segment of a strand of dental floss into an implement that can be inserted into the mouth and manipulated so as to properly control the application of the floss. These devices are commonly known as flossers. These devices provide a convenient mechanism for the flossing of teeth without the need for lengthy strands of floss. They also serve to more effectively reach into the spaces between the teeth so as to carry out flossing activities in a more effective manner. Flossers have also been combined to with toothpicks to form devices commonly known as floss toothpicks. These have become a common means of maintaining dental hygiene throughout the day, including after meals.

In the past, various patents have issued relating to such flosser implements. The earliest flosser apparatus that was revealed is in U.S. Pat. No. 2,187,899, issued on Jan. 23, 1940 to I. Henny. This patent describes a dental floss throw-away unit in which a single strand of thread extends between outwardly extending arms. A head is formed with the arms extending radially outwardly therefrom. The strand of floss extends in parallel relationship to the back of the head.

U.S. Pat. No. 2,648,341, issued on Aug. 11, 1953 to S. Moll teaches a dental floss holder which includes an elongated flexible member formed of plastic material. One end of the flexible member is rounded and provided with a transverse bore. A length of dental floss will extend through the transverse bore.

German Patent No. 29 23 057 teaches a dental floss applicator which includes a plurality of strands of floss which are far apart and extend in a plane which is perpendicular to the holder portion. Since the strands are not aligned with the shank portion of this flosser device, they are relatively difficult to apply as floss to one's teeth. The flosser is removably secured within a U-shaped head portion.

U.S. Pat. No. 4,280,518, issued on Jul. 28, 1981 to S. M. Gambaro teaches a tooth cleaning implement which includes an elongated member which has, at one end, a strand of dental floss tautly stretched thereacross. The opposite end of the elongated member is attached to a brush-like member which is used to facilitate the cleaning of teeth and dental bridges.

U.S. Design Pat. No. 276,088, issued on Oct. 23, 1984 to A. Fong describes a conventional flosser apparatus in which a single strand of floss is retained between a pair of arms extending outwardly of a head portion. A strand is connected to the head portion and extends so as to terminate at a pointed end.

U.S. Pat. No. 4,522,216, issued on Jun. 11, 1985, to R. L. Bunker describes a dental floss applicator which comprises a solid rectangular shaped body fitted with a pair of adjacent end arms forming a yolk arrangement in which the floss is drawn so as to form an X-shaped pattern. A small button fastener on each side of the applicator body permits the fastening of the floss after it has been stretched taut around the yolk.

German Patent No. 3,831,039 issued to H. Bauer describes a device for cleaning the narrow space between a bridge and the jaw. A pair of threads are connected to a guide. The threads are arranged in parallel to each other and are connected to each other by a number of parallel transverse threads.

U.S. Pat. No. 5,016,660, issued on May 21, 1991 to M. S. Boggs describes an automatic flossing tool having reciprocating tines supporting the flossing material and biased apart so as to assure proper tension on the flossing material. The device includes a means carried out by the tines for moving the flossing material between the tines and having a removable head so as to permit replacement of the head to provide sterile use for subsequent users.

U.S. Pat. No. 5,538,023, issued on Jul. 23, 1996 to Oczkowski et al., describes a tensioning dental flosser having a holder, a bow and a length of dental floss spanning the bow. A movable element is provided which can cause a portion of the floss holder to move and tighten the strand of floss so as to reduce the slack in the floss. U.S. Pat. No. 5,692,531, issued on Dec. 2, 1997 to I. S. Chodorow, describes a dual strand dental flosser having a body part, first and second spaced apart arms extending from the body part, a first strand of dental floss extending axially between the arms and a second strand of dental floss extending axially between the arms and generally parallel to the first strand of dental floss. A lever mechanism extends from one of the arms which is movable so as to be moved toward the body part. When this lever is moved toward the body part, the first and second strands will tighten. U.S. Pat. No. 5,829,458, issued on Nov. 3, 1998 to I. S. Chodorow, describes a dental floss holder of similar construction to that of U.S. Pat. No. 5,692,531. It shows a variety of other mechanisms that can be used for tightening the dental floss.

A nimeity of patented devices use a matchbook folded structure for supporting a variety of accessories for personal use that are in compact form and easy to carry. These patents include U.S. Pat. Nos. 1,832,604; 2,303,986; 2,336,234; 3,438,486 and 3,902,509. U.S. Pat. No. 1,832,604 to Wupper teaches a package containing any number of rows and files of rubber strands used as rubber dental cleaners. The patent is of interest for its teaching of individual, removable dental cleaners, the manner of packaging, namely the folded cardboard structure and the teaching away of the use of thread-like material, presumably dental floss.

U.S. Pat. Nos. 2,336,234 and 3,438,486 are of interest for teaching the use of a matchbook structure to support various personal care and other personal utility devices. The '234 patent teaches including matches along with a plurality of toothpicks.

None of the above patents or disclosures describe a business card advertising device incorporating a floss toothpick that may be readily produced and distributed. It is therefore desirable to provide such a device.

It is also desirable to provide a floss toothpick that includes a reflective portion to inspect the mouth and ensure that all extraneous debris has been sufficiently removed.

### SUMMARY

Accordingly, the primary object of the present invention is to provide a personal hygiene apparatus in matchbook form including an envelope-like structure, a plurality of dental cleaning devices that may be individually packaged and are detachably secured to the envelope.

The envelope includes a back panel, a flap, a bendable or foldable top, a base and a tab. The flap may be secured underneath the tab to retain the envelope in a closed position. Within the envelope is at least one dental cleaning device that may be attached to a comb or alternatively to the inside wall of the back panel. Optionally the dental cleaning devices may be individually wrapped. The dental cleaning devices may also optionally include a reflective surface.

It is therefore an object of the present invention to provide an improved dental floss toothpick and means of containing them.

It is a further object of the present invention to provide a flosser apparatus which is easy to use, relatively inexpensive, and easy to manufacture and assemble.

It is a further object of the present invention to provide a means for advertising combined with a dental hygiene device.

These and other objects and advantages of the present invention will become apparent from a reading of the attached specification and appended claims. There has thus been outlined, rather broadly, the more important features of the invention in order that the detailed description thereof that follows may be better understood, and in order that the present contribution to the art may be better appreciated. There are features of the invention that will be described hereinafter and which will form the subject matter of the claims appended hereto.

### BRIEF DESCRIPTION OF THE DRAWINGS

FIG. 1 is a front view of an open envelope in accordance with the principles of the present invention;

FIG. 2 is a back view of an open envelope in accordance with the principles of the present invention;

FIG. 3 is a perspective view of an envelope and dental cleaning devices in accordance with the principles of the present invention in an open position;

FIG. 4 is a perspective view of an envelope and dental cleaning devices in accordance with the principles of the present invention in a closed position;

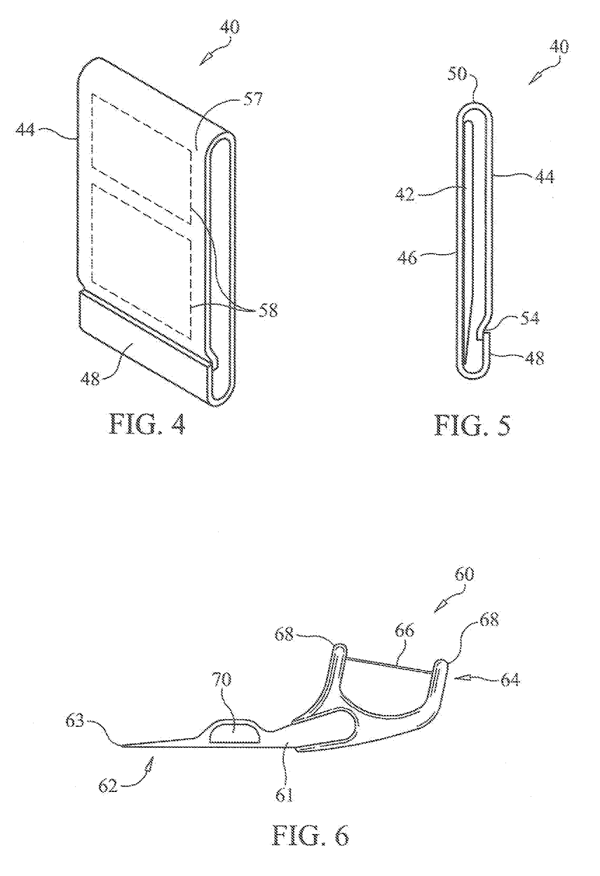

FIG. 3 is a side elevational view of an envelope and dental cleaning devices in accordance with the principles of the present invention;

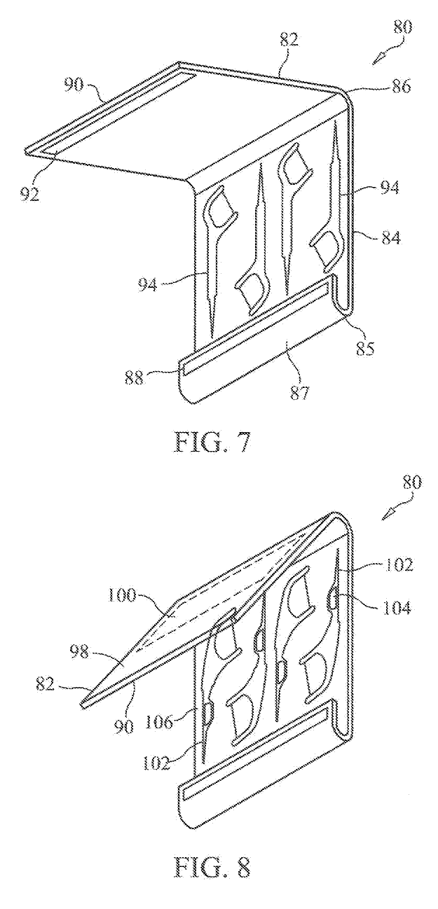

FIG. 4 is a top plan view of a dental cleaning device of the invention in accordance with the principles of the present invention;

FIG. 5 is a perspective view of an alternative envelope and dental cleaning devices in accordance with the principles of the present invention;

FIG. 6 is a perspective view of an alternative envelope and dental cleaning devices in accordance with the principles of the present invention.

### DETAILED DESCRIPTION

Before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

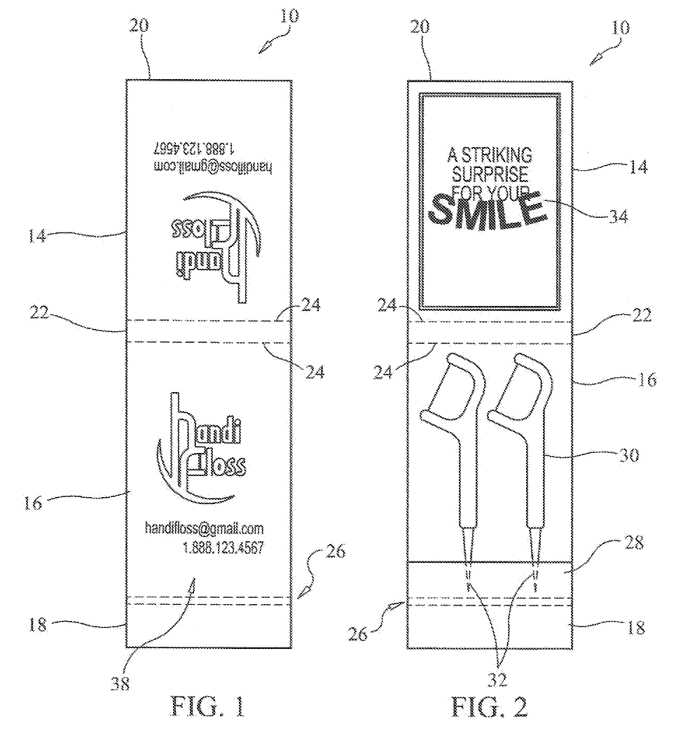

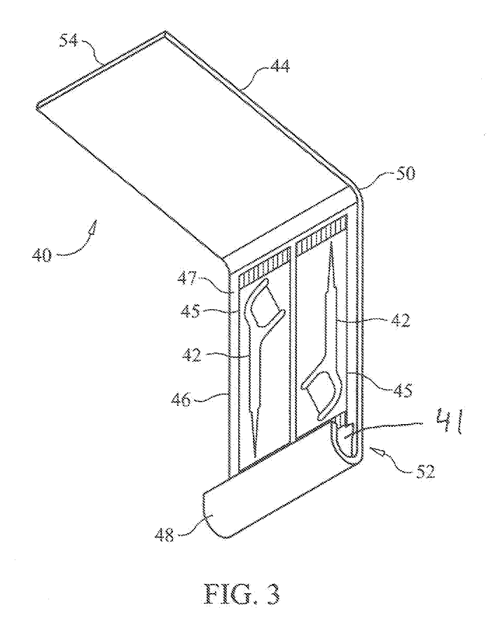

FIGS. 1 and 2 show a floss toothpick package comprised of an envelope 10 in accordance with the present invention. The envelope 10 may be formed from a singular unit of material, such as for example cardboard, that may be semi-rigid, planar and foldable. The envelope 10 comprises a front flap 14, a back panel 16 and a tab 18. It may be appreciated that this packaging design borrows some elements from a matchbook-like package, such that the front flap 14 and the tab 18 both fold over the back panel 16 and the leading edge 20 of the front flap 14 may be tucked underneath tab 18 to enclose the contents of the envelope. Optionally, a top panel 22 may be formed by the use of two creases 24 between the front flap 14 and the back panel 16. Optionally, tab 18 may also be folded over back panel 16 by use of two creases at the bottom 26 of the envelope 10. If a material that is not amenable to the formation of creases along which to fold in a pivotable fashion, a hinge device may be used in place of the creases illustrated in FIGS. 1 and 2. Alternatively, use of a more flexible or pliable material for the envelope may obviate the need for creases or hinges.

Located at the bottom of the envelope 10, an anchor strip 28 may be attached to the bottom of the back panel 16. The anchor strip 28 may be attached to the back panel only or may optionally also be secured to the flap 18 once the flap 18 is folded over. One or more floss toothpicks 30 may be detachably secured to the anchor strip 28 by means of perforated lines 32. In this embodiment, the package 10 includes two floss toothpicks 30, however, more or fewer may optionally be included. The entire package 10 may be sealed within a wrapper or the floss toothpicks 30 may be individually sealed within a wrapper, as is used to hermetically seal an object to maintain sterility and/or cleanliness. In this embodiment, floss toothpicks are enclosed within the envelope. However, other interdental cleaning devices may be used, such as for example toothpicks, interdental brushes, flossing devices, brushes, mints and dissolving mouthwash strips. Similarly, combinations of the above devices may be included within the package.

The exterior 38 of the envelope 10 may include external images, such as for example advertisements, trademarks, logos or other images. Similarly, the interior side 34 of the front flap or the inside of the back panel may include internal images.

FIGS. 3-5 show an embodiment of many aspects of the invention including envelope 40 having a matchbook-like structure and housing two floss toothpicks 42. Envelope 40 may have a substantially planar back panel 46 on which the floss toothpicks 42 lie. Envelope 40 also includes a substantially planar flap 44 connected to back panel 46 by folding top 50. Tab 48 extends from base 52 of back panel 46 and folds over so that it is parallel to back panel 46 and optionally holds an anchor strip 41, to which the wrappers of the floss toothpicks within envelope 40 are detachably secured. Flap 44 may be removably secured by inserting its leading edge 54 under tab 48.

On the outside of envelope 40 on an outer face 57 of flap 44 are two advertisements 58 Advertisements 58 may be printed onto outer face 57 or may be labels attached by adhesives. Alternatively, there may be only one advertisement 58 or a plurality. The back panel may similarly include such advertisements, not shown. Envelope 40 may be approximately the size of a business card and use of advertisements 58 may give envelope 40 the appearance of a business card. Alternatively any advertisements may be placed on outer face 57. Thus the envelope is adaptable for use in restaurants provided at the end of a meal optionally with a mint or similar object.

As smoking has become less prevalent, there is decreased demand for matchbooks. However, social awareness of dental hygiene has increased. Therefore the envelope 40 of the invention may readily replace matchbooks as a practical device provided to restaurant patrons that also serve the second purpose of matchbooks-to advertise. Envelope 10 may be made from pliable cardboard, paper or similar materials that would be suitable for a business card and/or a matchbook.

FIG. 3 shows both floss toothpicks enclosed within a wrapper 45. Wrapper 45 may be made of cellophane, foil, plastic or other suitable wrapping material. This ensures that the floss toothpicks 42 are clean prior to use. However, such packaging may not be necessary. Additionally, floss toothpicks 42 are removably attached to the inside surface 47 of back panel 46 by any reasonable means including adhesives or the like or mechanical means such as for example staples, crimps or clamps, or the like.

FIG. 6 shows a a dental floss toothpick 60 suitable for use with the invention. Floss toothpick 60 has a body 61 having a toothpick region 62 and a flossing region 64. Toothpick region 62 is elongate and comes to a point 63 making it suitable for removing debris from between teeth as a typical toothpick. Flossing region 64 includes two arms 68 anchoring a floss filament 66 therebetween and may be used as a typical flossing device known in the art. Body 61 also includes a reflective surface 70 that typically may consist of a minor. This allows an operator to view his or her teeth and determine what interdental debris exists and whether it has been sufficiently removed. The reflective surface 70 allows a person to avoid asking others to check his teeth for him. It may be desirable to include one or more reflective surfaces at one or more locations about the body 61.

FIG. 7 shows another embodiment of the invention comprising an envelope 80 having a more square shape. Envelope 80 has a flap 82 and a back panel 84 connected by foldable top 86. At base 85 of back panel 84 tab 87 folds over back panel 84 and has an adhesive strip 88. Adhesive strip 88 corresponds to adhesive strip 92 found at the leading edge 90 of flap 82. Adhesive strips 88 and 92 are removably engaged by means of their adhesive properties. This illustrates another method by which an envelope may be closed.

Envelope 80 also includes four floss toothpicks 94. Accommodation of these additional floss toothpicks 94 result in the more square geometry of envelope 80. In this embodiment, no packaging surrounds typical floss toothpicks 94.

FIG. 8 shows the envelope 80 having alternate floss toothpicks 102 each of which includes a reflective surface 104. floss toothpicks 102 are attached to a comb 106 to which they are removably attached. In this embodiment the floss toothpicks 102 and comb 106 are of a plastic material and connected in a manner similar to plastic model kit pieces are attached to a substrate. This holds the floss toothpicks 102 firmly in place until they are used. This also may facilitate mass production of envelopes containing floss toothpicks.

Whereas, the present invention has been described in relation to the drawings attached hereto, it should be understood that other and further modifications, apart from those shown or suggested herein, may be made within the spirit and scope of this invention. Descriptions of the embodiments shown in the drawings should not be construed as limiting or defining the ordinary and plain meanings of the terms of the claims unless such is explicitly indicated.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

1. An interdental cleaning device package comprising:
an envelope having a back panel, a front flap, and a tab;
one or more interdental cleaning devices detachably secured within the envelope

2. The interdental cleaning device package of claim 1 wherein the one or more dental floss cleaning devices comprise one or more floss toothpicks.

3. The interdental cleaning device package of claim 2 wherein the one or more floss toothpicks have a reflective surface.

4. The interdental cleaning device package of claim 2 further comprising an anchor strip, and wherein the one or more floss toothpicks are detachably secured to the anchor strip.

5. The interdental cleaning device package of claim 1 wherein the package includes one or more external images.

6. The interdental cleaning device package of claim 1 wherein the package includes one or more internal images.

7. The interdental cleaning device package of claim 1 wherein the one or more interdental cleaning devices are each enclosed in a wrapper.

8. The interdental cleaning device package of claim 7 further comprising an anchor strip, and wherein the wrappers enclosing each of the one or more interdental cleaning devices is detabchably secured to the anchor strip.

9. The interdental cleaning device package of claim 1 wherein the envelope is enclosed in a wrapper.

10. The interdental cleaning device package of claim 1 wherein the one or more interdental cleaning devices are detachably secured to the back panel.

11. A floss toothpick package comprising:
an envelope having a back panel, a front flap, and a tab;
an anchor strip between the front flap and the tab;
one or more floss toothpicks detachably secured within the envelope; and,
one or more external images on the envelope.

12. The floss toothpick package of claim 11 wherein the one or more floss toothpicks have a reflective surface.

13. The floss toothpick package of claim 11 wherein the one or more floss toothpicks each have an enclosing wrapper.

14. The floss toothpick package of claim 13 further comprising an anchor strip between the tab and the back panel, wherein the wrappers enclosing the one or more floss toothpicks are detachably secured to the anchor strip.

15. The floss toothpick package of claim 14 has one or more internal images.

16. The floss toothpick package of claim 15 wherein the one or more floss toothpicks comprise two floss toothpicks.

17. The floss toothpick package of claim 11 further comprising and anchor strip wherein the one or more floss toothpicks are detachably secured to the anchor strip.

18. The floss toothpick package of claim 11 wherein the envelope is enclosed in a wrapper.

19. The floss toothpick package of claim 11 wherein the envelope has one or more internal images.

20. A method for providing one or more packaged floss toothpicks comprising:
providing an envelope having a back panel, a front flap and a tab, wherein the front flap and the tab fold over the back panel and a leading edge of the front flap engages the tab;
providing an anchor strip between the tab and the back panel
providing one or more floss toothpicks detachably secured to the anchor strip and positioned between the front flap and the back panel.
